(19) United States
(12) Patent Application Publication
(10) Pub. No.: US 2024/0085580 A1
(43) Pub. Date: Mar. 14, 2024

(54) PROTON CT SYSTEM WITH IMPROVED PROTON ENERGY DETECTOR

(71) Applicants: INSTITUT DE FISICA D'ALTES ENERGIES ( IFAE)-THE BARCELONA INSTITUTE OF SCIENCE AND, Bellaterra( Barcelona) (ES); University of Geneva, Geneva 4 (CH); Birmingham University, Birmingham (GB)

(72) Inventors: Thorsten Lux, Barcelona (ES); Federico Joaquin Sanchez Nieto, Carouge (Genève) (CH); Tony Price, Birmingham (GB); César Jesús Valls, Barcelona (ES); Marc Granado González, Birmingham (GB)

(73) Assignees: INSTITUT DE FISICA D'ALTES ENERGIES ( IFAE)-THE BARCELONA INSTITUTE OF SCIENCE AND, Bellaterra ( Barcelona) (ES); University of Geneva, Geneva 4 (CH); Birmingham University, Birmingham (GB)

(21) Appl. No.: 18/463,971

(22) Filed: Sep. 8, 2023

Related U.S. Application Data
(60) Provisional application No. 63/405,282, filed on Sep. 9, 2022.

(51) Int. Cl. G01T 1/29; A61B 6/00; A61B 6/03; G01T 1/208

(52) U.S. Cl. CPC G01T 1/2985; A61B 6/032; A61B 6/584; G01T 1/208

(57) ABSTRACT

A Proton Computer Tomography (pCT) system comprising:
a position tracker, able to reconstruct the proton trajectory within the body;
an energy detector, able to reconstruct the proton energy;
   wherein, the position tracker is made up of four Depleted Monolithic Active Pixel Sensors (DMAPS) placed in pairs at either side of a phantom, and the energy detector is a Super Thin Range Telescope located downstream comprising a plastic-scintillator made of layers of thin polystyrene bars oriented in alternate axis, perpendicular to the proton beam.

### CROSS-REFERENCE TO RELATED APPLICATIONS

This application claims priority to U.S. Patent Application No. 63/405,282, filed on Sep. 9, 2022, which is herein incorporated it its entirety, including the appendix thereto.

### BACKGROUND

### Field of the Invention

The present invention relates to systems for providing high energy particle beams for treating cancer, more in particular, it relates to Proton Beam Therapy.

### Description of the Related Art

With an aging society, cancer is becoming a key social challenge in the next years. A method to treat cancer, especially those that are located at positions inside the body, which are difficult or impossible to remove by surgery as brain tumors for example, is Proton Beam Therapy (PBT). Proton Beam Therapy uses protons of up to 250 MeV to treat cancers and it was first available in a clinical setting in 1989, but is undergoing rapid expansion around the world in recent years. By the end of 2019 over 213,000 people have been treated globally with Proton Beam Therapy and there are over 100 facilities in operation with as many currently under construction or development.

Proton Beam Therapy exploits the characteristic dose deposition profile offered by massively charged particles where an increased dose is deposited at the end of the protons range, the so-called Bragg Peak, while minimizing dose to the healthy tissue before and after the tumor. The range of the proton and therefore the region over which it deposits the increased dose is set by the proton's initial kinetic energy as it enters the patient and the materials, which the protons interact with along their path. This capability to deposit a large amount of energy used to kill the tumor cells in a well-defined area while minimizing the radiation dose deposited in other tissue is the key motivation for PBT and makes this therapy approach superior to X-ray radiotherapy.

For the optimal treatment planning a high-quality 3D map of the body's Relative Stopping Power (RSP) is needed. The state-of-the-art technique is to obtain a computed tomography (CT) of the target using X-rays which measure the electron density of materials and to map the image into RSP. This procedure introduces uncertainties of around 3.5% corresponding to up to 7 mm uncertainty in range under real conditions, although under lab conditions and sample tissues values of 1.6% (0.7%) for single (dual) energy CT were achieved.

An uncertainty of up to 7 mm might have some serious consequences in respect of the treatment success rate. The beam might either destroy or damage healthy tissue since it penetrates too far into the body or part of a tumor might not be irradiated and the cancer could recover. Both cases are not desirable at all.

An interesting alternative approach is the direct use of a proton beam for this measurement. This could have various advantages over the currently used X-ray CT since an important fraction of the RSP uncertainty is coming from the fact that the data taken with X-rays needs to be carefully mapped to the RSP for protons with the risk of introducing mis-calibration in this process. In addition, it might be advantageously for logistical and time, which implies also possible cost savings, reasons to use the same facility for the proton CT as for the treatment.

A proton CT system consists of Si trackers in front and behind of the body/phantom followed by a residual energy detector (calorimeter) that measures the residual energy of the protons after crossing the body/phantom. The image achieved in this way will be repeated for a large number of rotation angles allowing to reconstruct a 3D image of the density distribution in the body. The principle of a proton CT device is sketched in FIG. 1

The beam used for proton therapy is a pulsed beam with a repetition rate of 72 or even 106 MHz and about 10\hat{ }9 protons/s. Ideally, to minimize the modification the beam parameters for the usage of the same facility for proton CT, the imaging device should be able to work with these beam parameters. Thus, a requirement for proton CT systems is to be able to take data with MHz rates and ideally handle several protons simultaneously.

While proton CT systems are currently not available for the clinical usage, significant steps have been already achieved by international research groups and consortia. For instance, the Loma-Linda/UCSC Phase-II scanner was able to produce images of a human-head size anatomy [1], and to experimentally demonstrate good imaging capabilities paired with measurement rates similar to 1 MHz [2]. The PRaVDA/OPTIMA collaboration introduced the possibility of using a full solid-state system for pCT, with good imaging results [3].

Patent application WO2015189602 discloses a pCT system with calorimeters based on Si strip detectors, which are sampling calorimeters that purely rely on the range measurement. The Si strip layers are interleaved with absorber material in which the protons lose their energy and eventually are stopped. The Si strip detectors are only providing the information that a particle has crossed the sensor. The absorber material is not sensitive and the energy measurement is exclusively based on the last absorber layer the proton has reached. This sandwich approach also leads to different densities along the proton path, which introduces uncertainties in the energy estimation, especially since the densities are not close to the one of water.

For commercial applications, a significant drawback of the Si strip approach is cost. To ensure a good range measurement, the absorber thickness must be of the order of 3 mm. This implies that for proton energies of up to 250 MeV around 80 to 90 Si strip layers would be needed corresponding to area coverage of more than 1 m².

### SUMMARY OF THE INVENTION

The system of the invention is based on scintillator bars that avoid these drawbacks at rates still close to 10⁸ protons/s. Such scintillator bars are fully sensitive and allow not only to measure the range, but also the amount of scintillation light produced, which provides an extra estimation of the energy deposition in the calorimeter. This improves the energy measurement specially for low energetic protons with E<120 MeV. This is not possible with a Si strip detector.

The scintillator material has also a density close to the one of water, reducing uncertainties from extrapolating from one density to water and thus achieving better performance than any sampling calorimeter for proton CT.

Furthermore, the detector material costs are reduced by a factor of 100 easily compared to a Si-based calorimeter.

### BRIEF DESCRIPTION OF THE DRAWINGS

To complete the description and provide for better understanding of the invention, a set of drawings is provided. Said drawings illustrate a preferred embodiment of the invention, which should not be interpreted as restricting the scope of the invention, but just as an example of how the invention can be carried out.

FIG. 1 shows the working principle of a Proton Computer Tomography system.

FIG. 2 is a schematic representation of the invention.

FIG. 3 shows two sliced sections of a pCT performed with the system of the invention using single proton events.

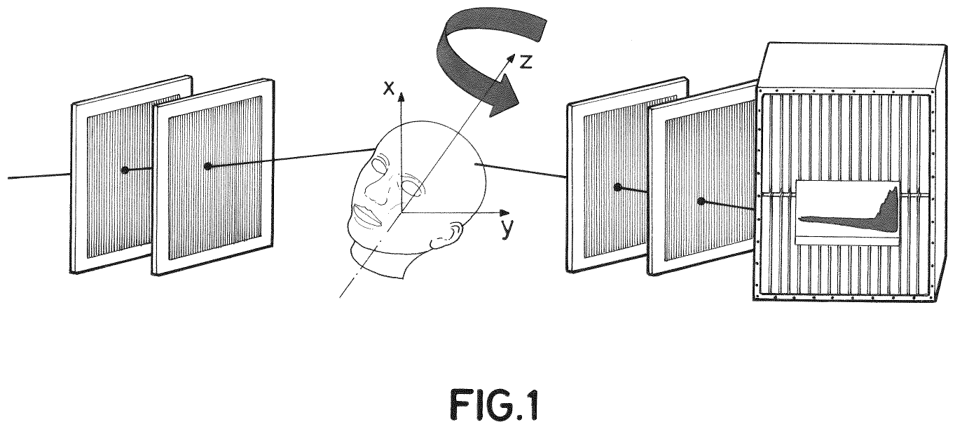

### DESCRIPTION OF THE PREFERRED EMBODIMENT

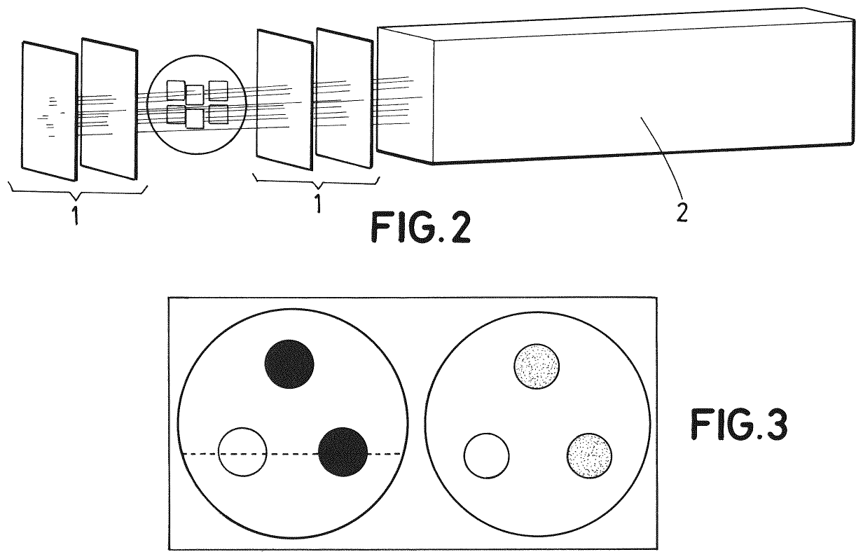

The Proton Computer Tomography (pCT) system of the invention is sketched in FIG. 2 and comprises:
A position tracker, able to reconstruct the proton trajectory within the body;
An energy detector, able to reconstruct the proton energy.

The position tracker is made up of four Depleted Monolithic Active Pixel Sensors (DMAPS) placed in pairs at either side of a phantom. The energy detector is a Super-Thin Range Telescope located downstream.

### DMAPS-Based Position Tracker:

Thanks to the fully depletion of the bulk DMAPS have a fast response and fast frame rate of 40 MHz. The tracker consists of four identical DMAPS, organized in two sub-trackers, front and back, each formed by a pair of DMAPS separated by 50 mm. We consider the gap distance between the front and back trackers to be of 150 mm, 100 μm thick with a shallow sensitive layer segmented forming an array of 2500×2500 silicon pixels of 40×40 μm² covering a total area of 10×10 cm2.

### Energy Tracker:

The energy detector is a plastic-scintillator range telescope consisting of layers made up of thin polystyrene bars oriented in alternate axis, perpendicular to the proton beam. In a particular embodiment, an EJ-200 plastic scintillator is used, which has a scintillation rise time of 0.9 ns, a decay time of 2.1 ns, and an attenuation length of 380 cm. Here, we consider a size of bars of 3×3×96 mm3, arranged in groups of 32 bars per layer. This provides a cross-section of 9.6×9.6 cm2, well matching the area of the DMAPS tracker. If necessary, the cross-section of the energy tracker could be easily scaled up by increasing the number of bars per layer. The length of the energy detector can be tuned to match the maximum beam energy, optimizing the production costs. With a length of 360 mm (120 layers), the detector is enough to stop protons of 240 MeV. The bars can be manufactured by plastic extrusion, and the outer layer produced by etching the bar surface. The bars are readout by a Silicon Photo Multiplier (SiPM) directly coupled to the scintillator bulk. In order to match the fast plastic response of the instrument, the fast output pulse shape from the Onsemi's MicroFJ SiPMs is used, providing a full waveform in the span of few nanoseconds.

Under this specifications, the light of two consecutive protons separated by a time span equal or higher to 10 ns can be well separated, and accordingly, the detector can reach an event rate equal or higher to 108 protons/s (100 MHz). Nonetheless, it must be noted that further reducing the proton's time gap to less than 10 ns with this same system could be possible, e.g. by doing a shape analysis of the waveform. Concerning the detector's geometry, using bars instead of layers means that the residual energy can be precisely reconstructed by range even if protons do not follow a perfectly straight trajectory. Secondly, if the beam has a typical spread comparable to the size of few of its bars, multiple protons can be tracked simultaneously when the time information is not enough to discriminate their trajectory. This has the advantage of further increasing the already high rate capability and reducing detector inefficiencies when the beam cannot be perfectly controlled to deliver a single proton per time frame.

Finally, the novelty of coupling the SiPM directly to the scintillator bulk will eliminate the necessity of introducing dead material inside sensitive volume of the detector, such as wavelength shifting fibers.

In a particular embodiment, the detector will have a 72×72×180 mm sensitive volume (corresponding to 1440 readout channels). A detector with such dimensions has enough depth to stop protons of 160 MeV covering the interesting energy range to study the energy resolution performance.

### Experimental Results:

The system of the invention was used for performing 3D pCT using a spherical phantom that consists in a 75 mm diameter sphere made of Perspex (PMMA) with six different cylindrical inserts 15 mm high with 15 mm diameter. The cylinders are placed in a three by three dis-position forming two equilateral triangle in two different planes placed 9 mm above and below the center. FIG. 3 shows two sliced sections of a pCT performed using single proton events. Each slice corresponds to the half height of the top (left image) and bottom (right image) sets of inserts. The different insert materials have been simulated to be equivalent to (from left to right): hard cortical bone, lung and air (left slice) and rib bone, water and adipose tissue (right slice). The measured mean values of the RSP for each insert have been computed by selecting the voxel values within the half diameter of each cylinder at six different layers around the center. An equivalent region has been selected to compute the RSP of the perspex frame. The RSP values extracted from FIG. 3 are presented in Table 2. True values have been computed using only true tracks and the true energy of the protons after passing through the phantom in order to provide a reference of the performance. All the reconstructed RSP values, except air, match the reference RSP values within 0.5%. The air value shows a larger relative discrepancy due to the small RSP of air, comparable to the measurement uncertainty.

| Material | RSP (Reco) | RSP (True) | % diff |
|---|---|---|---|
| Water | 0.992 ± 0.002 | 0.994 ± 0.002 | 0.201 |
| Air | 0.009 ± 0.002 | 0.008 ± 0.002 | −12.5 |
| Adipose | 0.916 ± 0.006 | 0.917 ± 0.005 | 0.109 |
| Rib bone | 1.325 ± 0.003 | 1.326 ± 0.001 | 0.075 |
| HC bone | 1.641 ± 0.003 | 1.646 ± 0.002 | 0.304 |
| Perspex | 1.144 ± 0.004 | 1.149 ± 0.002 | 0.455 |
| Lung | 0.302 ± 0.003 | 0.302 ± 0.002 | 0.000 |

The same 3D pCT image has been made using exclusively events with three simultaneous protons. Following a method analogous to that for the single proton events RSP results are presented in Table 2.

| Material | RSP (Reco 3p) | % diff (True) | % diff (reco 1p) |
|---|---|---|---|
| Water | 1.033 ± 0.002 | 3.924 | 4.133 |
| Air | 0.076 ± 0.006 | 850 | 744 |
| Adipose | 0.96 ± 0.02 | 3.60 | 3.71 |
| Rib bone | 1.34 ± 0.04 | 1.06 | 1.13 |
| HC bone | 1.66 ± 0.02 | 0.85 | 1.16 |
| Perspex | 1.14 ± 0.01 | −0.78 | −0.35 |
| Lung | 0.35 ± 0.02 | 15.89 | 15.89 |

Although a significant degradation is observed, especially for the materials with lower RSP, the results are of remarkable quality if one considers the fact that only 3-proton events are used. In a real-life situation, the beam settings could be configured to ensure a majority of 1-proton events. Multi-proton time frames, often unavoidable due to beam instabilities, would not account for inefficiencies, as in the other existing technologies. Instead, the multi-proton tracking features of the detector of the invention opens the door to develop reconstruction algorithms that associates different weights to each event depending on its reliability, with the goal to deliver a high quality pCT image.

Incorporated herein by reference is M. Granado-Gonzalez, et al., A novel range telescope concept for proton CT, Physics in Medicine and Biology, vol. 67, item 035013, 2022 (“Granado-Gonzales”). The authors of Granado-Gonzalez are the same as the inventors of this application.

As it is used herein, the term “comprises” and derivations thereof (such as “comprising”, etc.) should not be understood in an excluding sense, that is, these terms should not be interpreted as excluding the possibility that what is described and defined may include further elements, steps, etc.

On the other hand, the invention is obviously not limited to the specific embodiment(s) described herein, but also encompasses any variations that may be considered by any person skilled in the art (for example, as regards the choice of materials, dimensions, components, configuration, etc.) to be within the general scope of the invention as defined in the claims.

### REFERENCES

[1] T. Plautz et al., 200 MeV Proton Radiography Studies with a Hand Phantom Using a Prototype Proton CT Scanner, IEEE Transactions on Medical Imaging, vol. 33, no. 4, pp. 875-881, 2014.
[2] R. P. Johnson et al., A Fast Experimental Scanner for Proton CT: Technical Performance and First Experience with Phantom Scans, IEEE Transactions on Nuclear Science, vol. 63, no. 1, pp. 52-60, 2016.
[3] M. Esposito et al., PRaVDA: The first solid-state system for proton computed tomography, Physica Medica, vol. 55, pp. 149-154, 2018.

1. A Proton Computer Tomography (pCT) system comprising:
a position tracker;
an energy detector;
wherein the position tracker comprises a plurality of sensors;
wherein the energy detector comprises a telescope with a plastic-scintillator made of layers of polystyrene bars oriented in alternate axis, perpendicular to the proton beam.

2. The Proton Computer Tomography system of claim 1, wherein the position tracker is able to reconstruct the proton trajectory within the body.

3. The Proton Computer Tomography system of claim 1, wherein the energy detector is able to reconstruct the proton energy.

4. The Proton Computer Tomography system of claim 1, wherein the plurality is four, and wherein the four sensors are placed in pairs at either side of a phantom.

5. The Proton Computer Tomography system of claim 1, wherein the sensors are Depleted Monolithic Active Pixel Sensors.

6. The Proton Computer Tomography system of claim 1, wherein each of two halves of the plurality is placed at either side of the phantom.

7. The Proton Computer Tomography system of claim 1, wherein the telescope is a Super Thin Range Telescope.

8. The Proton Computer Tomography system of claim 7, wherein the Super Thin Range Telescope is located downstream.

9. The Proton Computer Tomography system of claim 1, wherein the telescope is located downstream.

10. The Proton Computer Tomography system of claim 1, further comprising a Si Photo Multiplier directly coupled to the scintillator.

11. The Proton Computer Tomography system of claim 1, wherein the detector has a 72×72×180 mm sensitive volume.

12. A method, comprising: performing a proton computer tomograhy with the Proton Computer Tomography system of claim 1, wherein the body's relative stopping power (RSP) of the protons is previously calculated with the same pCT system using a plurality of proton events.

13. A Proton Computer Tomography (pCT) system comprising:
a position tracker, able to reconstruct the proton trajectory within the body;
an energy detector, able to reconstruct the proton energy;
wherein, the position tracker is made up of four Depleted Monolithic Active Pixel Sensors (DMAPS) placed in pairs at either side of a phantom, and the energy detector is a Super Thin Range Telescope located downstream comprising a plastic-scintillator made of layers of polystyrene bars oriented in alternate axis, perpendicular to the proton beam.

14. The Proton Computer Tomography system of claim 13, further comprising a Si Photo Multiplier directly coupled to the scintillator.

15. The Proton Computer Tomography system of 13, wherein the detector has a 72×72×180 mm sensitive volume.

16. A method, comprising: performing a proton computer tomograhy with the Proton Computer Tomography system of claim 13, wherein the body's Relative Stopping Power (RSP) of the protons is previously calculated with the same pCT system using a plurality of proton events.
